(19) United States
(12) Patent Application Publication
(10) Pub. No.: US 2022/0314032 A1
(43) Pub. Date: Oct. 6, 2022

(54) DEVICES AND METHODS FOR APPLYING ULTRASOUND TO BRAIN STRUCTURES WITHOUT MAGNETIC RESONANCE IMAGING

(71) Applicant: BrainSonix Corporation, Sherman Oaks, CA (US)

(72) Inventors: Mark E. Schafer, Ambler, PA (US); Alexander Bystritsky, Sherman Oaks, CA (US)

(21) Appl. No.: 17/709,905

(22) Filed: Mar. 31, 2022

Related U.S. Application Data
(60) Provisional application No. 63/169,559, filed on Apr. 1, 2021.

(51) Int. Cl. A61N 7/00; A61B 8/06; A61B 8/08; A61B 8/00

(52) U.S. Cl. CPC A61N 7/00; A61B 8/06; A61B 8/488; A61B 8/42; A61B 8/4477; A61B 8/0808; A61N 2007/0026

(57) ABSTRACT

Ultrasound treatment of neurological conditions with energy levels that do not cause damage such as heating or cavitation. A method includes detecting a plurality of Doppler signals from a plurality of arteries near a brain structure, aligning an ultrasound transducer with the brain structure based on the plurality of Doppler signals, and applying ultrasound waves from the ultrasound transducer to the brain structure.

### CROSS-REFERENCE TO RELATED APPLICATIONS

This application claims the benefit of U.S. Provisional Application No. 63/169,559, filed Apr. 1, 2021, which is hereby incorporated by reference herein in its entirety.

### TECHNICAL FIELD

The technical field is ultrasonics and, more specifically, ultrasound treatment of neurological conditions with energy levels that do not cause damage such as heating or cavitation.

### BACKGROUND

In order to treat neurological problems with ultrasound, a certain level of ultrasound energy needs to be delivered to a specific area in the brain. Ultrasound has to be delivered through the bone of the skull and focused on the desired region within the skull. The reliable positioning and aiming of the transducer on the patient's skull is part of the overall treatment process. Treatments are typically carried out using a Magnetic Resonance Imaging (MM) system to image the brain and show the operator the region to be treated.

The transducer, also known as a probe or ultrasound transmitter, should be easily positioned by an operator, preferably without tools. The positioning system should allow for the transducer orientation to be easily adjusted. The transducer orientation should be readily apparent to the operator to easily determine the direction of the ultrasound beam, where the focal region of the transducer will intersect brain tissue. The transducer should also be easily coupled to the patient's skin surface, so that the ultrasound energy will pass unimpeded from the transducer into the patient's tissue. The entire system should be simple and intuitive to operate.

However, in many instances, it is not possible to use an MRI to image the patient's brain, or to provide real-time position or alignment feedback. This may be due to cost, complexity, or convenience to the patient.

Devices and methods for positioning an ultrasound transducer to apply ultrasound energy to, for example, insonify specific regions of the brain without the use or assistance of an Mill are needed.

### SUMMARY

In an embodiment, a method comprises detecting a plurality of Doppler signals from a plurality of arteries near a brain structure, aligning an ultrasound transducer with the brain structure based on the plurality of Doppler signals, and applying ultrasound waves from the ultrasound transducer to the brain structure.

In an embodiment, a system comprises an ultrasound transducer configured to generate ultrasound waves, a computing device, and a computer-readable storage medium comprising a set of instructions that upon execution by the computing device cause the system to detect a plurality of Doppler signals from a plurality of arteries near a brain structure, align the ultrasound transducer with the brain structure based on the plurality of Doppler signals, and apply the ultrasound waves from the ultrasound transducer to the brain structure.

### BRIEF DESCRIPTION OF THE DRAWINGS

The accompanying drawings, which are incorporated in and constitute a part of this specification, illustrate various embodiments of the invention and, together with a general description of the invention given above and the detailed description of the embodiments given below, serve to explain the embodiments of the invention. In the drawings, like reference numerals refer to like features in the various views.

FIG. 1 is a schematic representation of transducers placed on each the side of the skull of a human patient.

FIG. 2 is a schematic representation of the side view of a human skull, denoting the region of thin bone known as the temporal window.

FIG. 3 is a schematic representation of the position of the amygdalae within the skull. There are two amygdala locations on either side of the midline.

FIG. 4 is a schematic view of an exemplary computer system that may be used in conjunction with embodiments of the invention.

### DETAILED DESCRIPTION

Specific conditions and ailments can be treated, or potential conditions and ailments can be prevented, by insonifying the amygdala region of the brain. Embodiments disclosed herein may facilitate proper aiming of ultrasound energy from a transducer placed externally on the patient's head, towards the amygdala region, without the use of prior MRI/CT imaging or concurrent MRI guidance. The aiming of the ultrasound energy may be done using only the ultrasound transducer(s) and the ultrasound drive, receive, and processing electronics described herein.

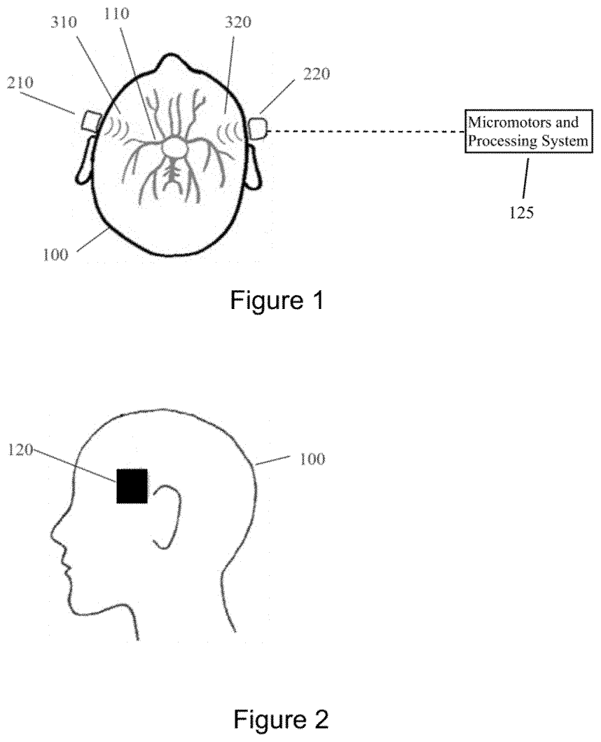

FIG. 1 is a schematic representation of transducers 210 and 220 placed on each side of the skull 100 of a human patient. This demonstrates the overall geometry and application of the embodiments described herein. This is the transverse plane of the skull 100. Each transducer 210 and 220 emits ultrasound waves 310 and 320, respectively. Also visible in FIG. 1 is a representation of the major arteries 110 within the brain 130.

Transducers 210 and 220 are placed at the region of the temples of the skull 100, where the skull bone is thinnest and most uniform. This region, 120, as depicted in FIG. 2, is called the temporal window because it affords an acoustic “window” through with ultrasound signals more easily pass.

The technique of TransCranial Doppler, or TCD, uses ultrasound transducers placed on the temporal window 120 in order to determine the bloodflow in the arteries of the brain 110. By locating the regions with Doppler bloodflow signals, at the correct depth, the transducers 210 and 220 can be aimed at different arterial structures within the brain.

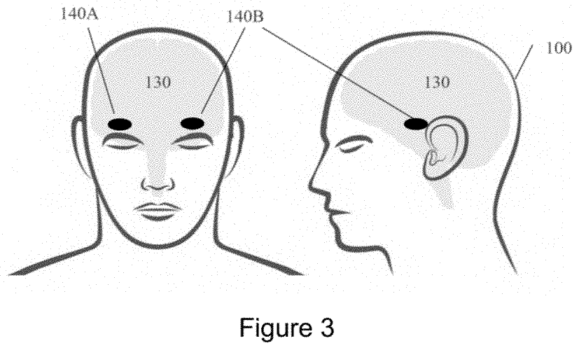

The amygdalae 140A and 140B are two almond shaped structures within the temporal lobes, and are involved in the processing of memory, decision-making, and emotional responses such as anxiety and aggression. By treating the amygdalae 140A, 140B, with the appropriate level of ultrasound energy 310, 320 from transducers 210 and 220, the emotional state of the patient can be affected, influenced, controlled, changed, heightened, lowered, and/or varied. The specific conditions include, but are not limited to Anxiety, Generalized Anxiety Disorder, Post-traumatic Stress Disorder, and Insomnia. Comparing FIG. 2 and FIG. 3, the amygdalae are positioned essentially directly in line with the temporal window 120, and also in the region of the major cerebral arteries 110.

The anterior choroidal artery is the preterminal branch of the internal carotid and provides blood supply to the amygdala. This artery arises near the junctures of the internal carotid artery, the middle cerebral artery, the posterior communicating artery, and the anterior cerebral artery. Those latter two arteries comprise part of the Circle of Willis. All such arteries can be located using Doppler flow techniques. The amygdalae are positioned near this conflux of arteries.

In practice, transducers 210 and 220 are initially used for detection of Doppler bloodflow signals from cerebral arteries 110. The transducers 210 and 220 are generally placed on the temporal window region 120 on each side of the skull 100, and are aimed inwards towards the centerline of the brain 130. By angulating the transducers 210 and 220, and using the appropriate time gate window function on the Doppler equipment, the operator can aim the transducers at different specific arteries. TransCranial Doppler (TCD) may be performed at 2 MHz, although it can also be performed at other frequencies. Lower frequencies may incur less attenuation/absorption in round trip transit from the transducer to the artery; higher frequencies may provide more precision in focusing. However, because only general positioning is required as described herein, the choice of the exact frequency may be determined by the system designer, taking into account other factors such as transducer cost.

By using the Doppler signals from the arteries near the amygdalae, the transducers 210 and 220 may be aligned such that they are aimed at the amygdalae, without the need for other imaging or guidance techniques, such as MRI or external optical, magnetic, acoustic, or mechanical alignment devices. The alignment of the transducers towards the amygdalae provides the necessary external positioning information. The Doppler range gate then can be used to determine the distance from the transducer to the amygdala region, should this information be required. The amygdalae are generally only a few centimeters below the skin surface, on the order of 4-5 centimeters, depending upon the patient. The cross-sectional area of the ultrasound beam, shown schematically as 310 and 320, may be on the order of 10 millimeters to 20 millimeters in order to encompass the entire amygdala from either side of the skull 100.

Once the transducers 210 and 220 are properly aligned at the amygdala 140B and 140A respectively using the Doppler signals, the transducers are subsequently used to insonify the respective amygdala with ultrasound in a pulse delivery manner which provides a neuro-modulatory effect. The pulse delivery manner may comprise pulsed signals with a range of pulse durations, pulse repetition frequencies (and by combination of the two, a range of duty factors), as well as drive frequency and amplitude. Additional neuro-modulatory pulse patterns may be determined to be effective for the specific patient being treated. The ultrasound stimulation may cause micro-mechanical deformations of the nerve tissue, thus inducing a mechanical transduction effect which either inhibits or excites the nerves. This action then may lead to an overall change in the patient's mood, or psychological or mental state.

The transducers 210 and 220 may be of several designs. In an embodiment, the transducers are each comprised of a single element Doppler transducer, and an annular treatment/stimulus/inhibition transducer surrounding the Doppler transducer. In another embodiment, the treatment transducer is in the central circular region, and the TCD transducer is in an annular configuration. In a third embodiment, a single transducer is used for both purposes. In this case, the operating frequency must be a compromise between the Doppler frequency and the treatment frequency. The Doppler frequency for TCD may be around 2 MHz, and the treatment frequency may be between 500 and 750 kHz, although other frequencies may be employed. In an embodiment, a single transducer may be operated in the fundamental frequency of 650 kHz for treatment, and may be operated at the 3^{rd} harmonic frequency of 1950 kHz for Doppler. In another embodiment, the same dual-frequency approach may be used, with the transducer operated at 666 kHz for treatment and 2 MHz for TCD.

In another embodiment, the non-treatment transducer comprises an imaging array, and the Doppler interrogation of the cerebral vasculature includes Color Doppler imaging. This may permit rudimentary direct imaging of the amygdala region.

In another embodiment, the treatment transducer comprises an annular array, such that it can be electronically focused to the depth of the amygdala, which is determined by the Doppler range gate method, or the image methods disclosed. The annular array may permit the focal depth to be electronically adjusted to provide maximum ultrasound energy at the required depth, while minimizing the energy elsewhere in the skull.

Other combinations of the transducer design may be used, mixing and matching various designs, including phased array, linear array, circular array, single element, two dimensional arrays, annular arrays, segmented transducers, and multifrequency transducers.

In another embodiment, multiple transducers on each side of the skull 100, arrayed in parallel to transducers 210 and 220 shown, may be used. Each transducer within the group may be used separately, and the transducer which produced the best signal match (most appropriate Doppler signal at the appropriate depth from the transducer), is used for subsequent treatment.

In another embodiment, the focal diameter of the TCD transducer configuration is smaller than the focal diameter of the treatment transducer configuration. The treatment transducer configuration should have a focal diameter of 10 millimeters to 15 millimeters, which is the projected size of the amygdala in the direction of the treatment transducer. In another embodiment, the focal diameter of the TCD transducer configuration is the same size as the focal diameter of the treatment transducer configuration.

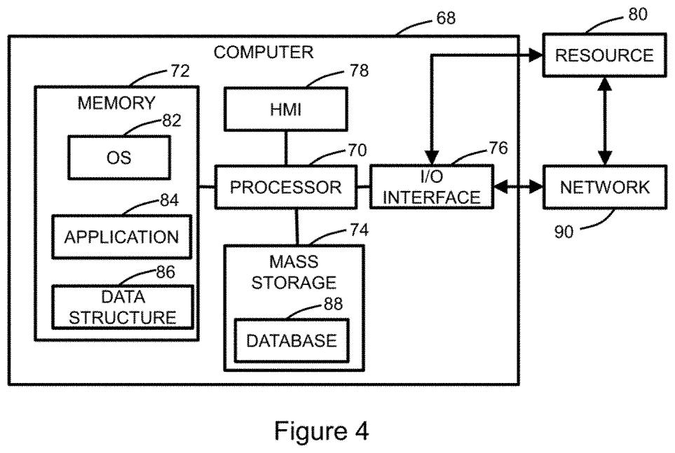

The angular positioning of the transducers 210 and 220 may be done manually, using a frame to maintain spatial position and contact between the transducers 210 and 220 and the skull 100. In another embodiment, the positioning may be adjusted by external micromotors, which occur under the control of a computer system (e.g., FIG. 4) or other processing system, as generally indicated by reference numeral 125. The processing system may cause the angle of the transducers 210 and 220 to change, while adjusting the range gate of the Doppler system, to effectively map out the bloodflow profile within the brain 130. This may also be done using Color Flow mapping. Computer analysis software, such as neural nets or machine learning algorithms, can be used to determine, based on prior “learning” trials, the optimal angular position of the transducer for insonifying the amygdalae.

The micromotors for positioning can be any of a number of different types, including stepper motors, linear motors, solenoids, piezoelectric motors, with or without gearing mechanisms.

In another embodiment, the array of multiple transducers on each side of the skull 100 may be sequentially used with the same type of analysis software to determine the optimal treatment transducer. In this case, each of the multiple transducers is angled slightly differently from one another, such that their beam patterns at the depth of interest of the amygdala only slightly overlap. In this way, selecting different transducers is functionally equivalent of angularly adjusting a single element transducer. The person of skill in the art may decide the best approach, with the tradeoff of the complexity of the micromotor versus the cost of additional transducers.

The embodiments described herein eliminate the need for MRI imaging, or another positioning/imaging system, for guidance before or during treatment procedures involving the use of ultrasound and the amygdala. The transducers 210 and 220 may be aligned towards the target tissue 140A and 140B within the skull 100 without a separate imaging system, which creates the option of a treatment system that is completely self-contained, portable, and patient centric. In addition, the patient is not subjected to ionizing radiation (CT) or high magnetic fields (MRI).

When used with a micromotor or multiple transducer alignment system, treatments can be automated, thereby reducing the skill requirement of the treatment operator, and thus lowering costs.

By using Doppler signals as the initial target, the devices described herein can adapt to the structural anatomy of the specific patient being treated, without the need for other imaging modalities. This also avoids the need to accurately transfer anatomical information from a large and expensive imaging system, such as an Mill or CT imaging system, to the ultrasound transducer positioning system.

The embodiments described herein are not complex, expensive, or cumbersome, and are amenable to use outside of facilities with sophisticated equipment. The embodiments described herein may provide an inexpensive, robust approach that removes limitations on the use of ultrasound for the treatment, prevention, or amelioration of brain or neurological conditions or diseases.

The embodiments described herein can be completely self-contained with regard to ultrasound Doppler and treatment transducers and electronics. With optimization, the embodiments described herein can potentially be battery operated, as the total energy required for neuromodulation is not high.

Based on the signal strength of the TCD (not velocity reading), the attenuation of the ultrasound signal in the skull 100 and brain 130 can be estimated, and the drive signal to the treatment transducer configuration can be adjusted to neither overtreat nor undertreat the patient, applying sufficient ultrasound energy to cause neurostimulation. This can be done either using the transducers 210 and 220 independently (each doing an estimate only for one side), or using the transducers 210 and 220 in a coordinated fashion, with signals that cross over from one side of the skull 100 to the other being used as part of the signal estimation process.

In an embodiment, the operation may be switched back and forth between TCD and treatment, and changes in cerebral bloodflow may be used as an indication that the treatment is having an effect. This contrasts with other means of determining whether the ultrasound is having an effect on cerebral tissue, namely functional Mill and MM arterial spin labeling (ASL), both of which require the patient to be located in an MRI.

The systems described herein may be conveniently combined with other technologies that influence mood, emotion, fear, or anxiety, etc., such as visual or auditory signals applied to the patients eyes or ears. Such combinations may permit further enhancement of the influences of the visual and auditory signals, by directly stimulating the brain region responsible for the emotions or feelings to be influenced.

With the location information provided by the embodiments described herein, it is also possible to target other brain structures which are in a fixed position relative to the arterial structures described. Other brain structures near the amygdala, for instance but not limited to, the hippocampus, may be targeted without the need for MRI or CT guidance. Using TCD with positional information, or using Color Doppler imaging techniques, may allow for targeting these other brain structures conveniently without MRI or CT guidance.

As shown in FIG. 4, an exemplary computer system 68 may be configured to function as processing system, to interface with and control the transducers 210, 220, to interface with and control the micromotors for positioning the transducers 210, 220, etc. The computer system 68 may include a processor 70, a memory 72, a mass storage memory device 74, an input/output (I/O) interface 76, and a Human Machine Interface (HMI) 78. The computer system 68 may also be operatively coupled to one or more external resources 80 via the I/O interface 76. External resources 80 may include, but are not limited to, servers, databases, mass storage devices, peripheral devices, cloud-based network services, or any other suitable computer resource that may be used by the computer system 68.

The processor 70 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in the memory 72. The memory 72 may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, or any other device capable of storing information. The mass storage memory device 74 may include data storage devices such as a hard drive, optical drive, tape drive, non-volatile solid state device, or any other device capable of storing information.

The processor 70 may operate under the control of an operating system 82 that resides in the memory 72. The operating system 82 may manage computer resources so that computer program code embodied as one or more computer software applications, such as an application 84 residing in memory 72, may have instructions executed by the processor 70. In an alternative embodiment, the processor 70 may execute the application 84 directly, in which case the operating system 82 may be omitted. One or more data structures 86 may also reside in memory 72, and may be used by the processor 70, operating system 82, or application 84 to store or manipulate data. The application 84 may include modules with instructions for controlling the operation of the transducers 210, 220, controlling and driving the micromotors to position the transducers 210, 220, etc.

The I/O interface 76 may provide a machine interface that operatively couples the processor 70 to other devices and systems, such as the one or more external resources 80, and other hardware such as the transducers 210, 220 and micromotors. The application 84 may thereby work cooperatively with the external resources 80 by communicating via the I/O interface 76 to provide the various features, functions, applications, processes, or modules comprising embodiments of the invention. The application 84 may also have program code that is executed by the one or more external resources 80, or otherwise rely on functions or signals provided by other system or network components external to the computer system 68. Indeed, given the nearly endless hardware and software configurations possible, persons having ordinary skill in the art will understand that embodiments of the invention may include applications that are located externally to the computer system 68, distributed among multiple computers or other external resources 80, or provided by computing resources (hardware and software) that are provided as a service over a communication network 90, such as a cloud computing service.

The HMI 78 may be operatively coupled to the processor 70 of computer system 68 in a known manner to allow a user to interact directly with the computer system 68. The HMI 78 may include video or alphanumeric displays, a touch screen, a speaker, and any other suitable audio and visual indicators capable of providing data to the user. The HMI 78 may also include input devices and controls such as an alphanumeric keyboard, a pointing device, keypads, pushbuttons, control knobs, microphones, etc., capable of accepting commands or input from the user and transmitting the entered input to the processor 70.

A database 88, which may reside on the mass storage memory device 74, may be used to collect and organize data used by the various systems and modules described herein. The database 88 may include data and supporting data structures that store and organize the data. In particular, the database 88 may be arranged with any database organization or structure including, but not limited to, a relational database, a hierarchical database, a network database, or combinations thereof. A database management system in the form of a computer software application executing as instructions on the processor 70 may be used to access the information or data stored in records of the database 88 in response to a query, where a query may be dynamically determined and executed by the operating system 82, other applications 84, or one or more modules.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
detecting a first plurality of Doppler signals from a plurality of arteries near a brain structure;
aligning a first ultrasound transducer with the brain structure based on the first plurality of Doppler signals; and
applying ultrasound waves from the first ultrasound transducer to the brain structure.

2. The method of claim 1 wherein the brain structure is an amygdalae.

3. The method of claim 1 further comprising:
determining a distance from the first ultrasound transducer to the brain structure based on the first plurality of Doppler signals.

4. The method of claim 1 wherein the first ultrasound transducer is used to generate the first plurality of Doppler signals.

5. The method of claim 1 wherein a second ultrasound transducer is used to generate the first plurality of Doppler signals.

6. The method of claim 1 wherein applying the ultrasound waves to the brain structure comprises:
insonifying the brain structure with the ultrasound waves applied in a pulsed delivery manner.

7. The method of claim 1 wherein the first ultrasound transducer is placed externally to a skull of patient.

8. The method of claim 1 further comprising:
detecting a second plurality of Doppler signals from the plurality of arteries near the brain structure.

9. The method of claim 8 wherein the first plurality of Doppler signals are used to determine a first blood flow in the plurality of arteries, the second plurality of Doppler signals are used to determine a second blood flow in the plurality of arteries, and further comprising:
comparing the second blood flow to the first blood flow.

10. The method of claim 1 wherein the ultrasound waves applied to the brain structure impart a neuro-modulatory effect on the brain structure.

11. A system comprising:
a first ultrasound transducer configured to generate ultrasound waves;
a computing device; and
a computer-readable storage medium comprising a set of instructions that upon execution by the computing device cause the system to:
detect a first plurality of Doppler signals from a plurality of arteries near a brain structure;
align the first ultrasound transducer with the brain structure based on the first plurality of Doppler signals; and
apply the ultrasound waves from the first ultrasound transducer to the brain structure.

12. The system of claim 11 wherein the computer-readable storage medium further comprises a second set of instructions that upon execution by the computing device further cause the system to:
determine a distance from the first ultrasound transducer to the brain structure based on the first plurality of Doppler signals.

13. The system of claim 11 wherein the first ultrasound transducer is used to generate the first plurality of Doppler signals.

14. The system of claim 11 further comprising:
a second ultrasound transducer configured to generate the first plurality of Doppler signals.

15. The system of claim 14 wherein the second ultrasound transducer is an annular structure, and the first ultrasound transducer is positioned inside the annular structure.

16. The system of claim 14 wherein the first ultrasound transducer is an annular structure, and the second ultrasound transducer is positioned inside the annular structure.

17. The system of claim 11 wherein the ultrasound waves applied in a pulsed delivery manner to insonify the brain structure.

18. The system of claim 11 wherein the first ultrasound transducer is placed externally to a skull of patient.

19. The system of claim 11 wherein the computer-readable storage medium further comprises a second set of instructions that upon execution by the computing device further cause the system to:
detect a second plurality of Doppler signals from the plurality of arteries near the brain structure.

20. The system of claim 19 wherein the first plurality of Doppler signals are used to determine a first blood flow in the plurality of arteries, and the second plurality of Doppler signals are used to determine a second blood flow in the plurality of arteries that is compared to the first blood flow.
